(19) United States
(12) Patent Application Publication
(10) Pub. No.: US 2023/0408470 A1
(43) Pub. Date: Dec. 21, 2023

(54) SYSTEM AND METHOD FOR MONITORING PURITY OF FLUID

(71) Applicants: You Hin Pang, Singapore (SG); Agile Biz Tech Pte Ltd, Singapore (SG)

(72) Inventors: You Hin Pang, SINGAPORE (SG); Hock Wah Ng, SINGAPORE (SG); Hwee Lay Wendy Kho, SINGAPORE (SG); Boom Tom Lim, SINGAPORE (SG)

(21) Appl. No.: 18/212,469

(22) Filed: Jun. 21, 2023

(30) Foreign Application Priority Data
Jun. 21, 2022 (SG) 102250247Y

(51) Int. Cl. G01N 33/00; G01N 1/38

(52) U.S. Cl. CPC G01N 33/0016; G01N 1/38; G01N 33/0073; G01N 33/0063

(57) ABSTRACT

A system and a corresponding method for monitoring the purity of fluid are described. The system includes a duct (2) connecting to a fluid source (1), which includes a first valve (212) for controlling the fluid flowing from the fluid source (1) into the duct (2), or portions thereof, as a sample, a purity sensor (22) disposed downstream of the first valve (212), and one or more pressure regulators for controlling pressure within the duct (2), or portions thereof. The duct (2), or portions thereof, is of an environment with a desired pressure for measuring a purity level of the sample by the purity sensor (212).

### CROSS-REFERENCE TO RELATED APPLICATION

The instant application claims priority to Singapore Patent Application Serial No. 10202250247Y filed Jun. 21, 2022, the entire specification of which is expressly incorporated herein by reference.

### FIELD OF THE INVENTION

The invention relates to purity monitoring. More particularly, the invention relates to a system and a method for monitoring the purity of fluid that involves the creation of an environment that is pressure regulated.

### BACKGROUND OF THE INVENTION

Purity monitoring refers to a quality control procedure in which the purity of a fluid, being either in liquid or gaseous phase is monitored. In particular, such a procedure is of utmost importance to ensure that fluid has a purity level that meets purity requirements so that it is suitable for its intended application. Use cases may be, but shall not be limited to, the production of a gas laser, refrigerants, flame retardants, cleaning agents, or the like. Should the purity level of the fluid fail to meet purity requirements, the impure fluid may seriously disrupt its intended application.

There are a few disclosed technologies over the prior art relating to the monitoring of purity of fluid. Among them is CN205746026U, which discloses a kind of system that monitors the purity of gas and directs it to be stored in different containers depending on the measured purity level of the gas.

Another disclosed technology is US20060204910A1, which discloses a kind of fuel injection system that monitors the purity of fluid. In particular, the fluid is directed into a branched pipeline for its purity level or concentration level to be measured through a plurality of sensors. Depending on the measured purity level or concentration level, a pressure regulator changes the pressure of the fluid within the main pipeline accordingly for the control of a combustion process.

Even so, the purity monitoring systems of CN205746026U and US20060204910A1 do not take into consideration for their purity sensors to operate in an optimal manner, nor do they provide considerations for a system that can accommodate fluid in a liquid or gaseous phase. Accordingly, it would be desirable to have a monitoring system that takes into consideration and creates an environment suitable for the sensor.

### SUMMARY OF THE INVENTION

The main objective of the present invention is to provide a system and a method for monitoring the purity of fluid that involves the creation of an environment that regulates the pressure of the fluid. The fluid is preferably gaseous carbon dioxide or liquid carbon dioxide. To achieve this objective, there is a computerised duct that takes a fluid portion from a fluid source as a sample, and further creates an environment in which the pressure of the fluid is regulated, so that its purity sensor measures a purity level of the sample. The purity level of the sample shall correspond to the purity level of fluid flowing within the fluid source. There is also a phase conversion unit that may convert the sample from being in a liquid phase into a gaseous phase. Advantageously, the present invention possesses portability, as it is easily incorporated in various points along the body of the fluid source to be used. Also, the present invention provides the monitoring of fluid in real-time, especially for critical applications without assuming on purity uniformity of the fluid or relying on a static testing event. Also, the present invention provides a kind of system and method that is non-destructive toward the fluid. Also, the present invention provides an interlinked and holistic approach to plant processes that adheres to the trend toward Industry 4.0. Also, the present invention is capable of being configured to measure the purity level of the fluid in a gaseous phase or liquid phase. Finally, the purity sensor, in conjunction with a computer of the system, is capable of identifying and differentiating constituents within the fluid whether they are in a solid, liquid or gas phase.

The invention intends to disclose a system for monitoring purity of fluid, comprising a duct connecting to a fluid source, which includes a first valve for directing the fluid flowing from the fluid source into the duct, or portions thereof, as a sample, a purity sensor disposed downstream of the first valve, and one or more pressure regulators for controlling pressure within the duct, or portions thereof. The duct, or portions thereof, is of an environment with a desired pressure for measuring a purity level of the sample by the purity sensor.

Preferably, the duct further comprises a phase conversion unit located upstream of the purity sensor for converting the sample from a liquid phase into a gaseous phase.

Preferably, the pressure regulators include any one or a combination of a first pressure regulator disposed downstream of the phase conversion unit for regulating the pressure of the sample after it passes through the phase conversion unit, and a second pressure regulator disposed upstream of the purity sensor, for regulating the pressure of the sample before it passes through the purity sensor.

Preferably, wherein the desired pressure ranges from 0.9 bar to 5 bar.

Preferably, the duct further comprises a second valve located downstream of the purity sensor for purging the sample therefrom.

Preferably, the system further comprising at least one processor that operates a combination of modules that include a master control module for at least controlling either one or a combination of the first valve, the second valve, a recorder module for recording the purity level of the sample and storing it in a memory unit, and a comparison module for comparing the purity level of the sample with a threshold.

Preferably, the processor further operates one or a combination of modules that include a timing module, for controlling the frequency of monitoring, a security module, for protecting the system from unauthorized use, a user interface module, for compiling purity levels that were recorded through the recorder module for them to be displayed on a human-machine interface unit.

Preferably, the system further comprises an alert unit that produces either one or both audio and visual stimuli when the purity level of the sample is below the threshold.

Preferably, wherein the duct further comprises a return structure provided upstream of the first valve and connected upstream of the fluid source that allows fluid that entered the duct to return to the fluid source.

Preferably, the fluid is either liquid carbon dioxide or gaseous carbon dioxide.

The invention also intends to disclose a method for monitoring purity of fluid, comprising the steps of directing the fluid from a fluid source into a duct of a system for monitoring the purity of fluid, as a sample, and measuring a purity level of the sample within the duct by a purity sensor. The duct, or portions thereof, is of an environment with a desired pressure for measuring the purity level of the sample by the purity sensor.

Preferably, the method further comprises the step of converting the sample within the duct from a liquid phase into a gaseous phase by a phase conversion unit prior to measuring the purity level of the fluid by the purity sensor.

Preferably, the method further comprises either one or both the steps of regulating the pressure of the sample after it passes through the phase conversion unit, by a first pressure regulator, and regulating the pressure of the sample before it passes through the purity sensor by a second pressure regulator.

Preferably, the desired pressure of the said method ranges from 0.9 bar to 5 bar.

Preferably, the method further comprises the step of purging the sample from the duct through a second valve upon measuring the purity level of the fluid.

Preferably, the method further comprises the steps of controlling either one or both the first valve and the second valve to open or shut, through a master control module, recording the purity level of the sample, through a recorder module, and comparing the purity level of the sample with a threshold, through a comparison module.

Preferably, the step of comparing the purity level of the sample with a threshold further comprises the steps of determining that the purity level of the sample is below the threshold, producing either one or both audio and visual stimuli using an alert unit, and preventing further flow of the fluid into the fluid source.

Preferably, the method further comprises the step of compiling the purity levels that were recorded by the recorder module to be displayed on a human-machine interface unit, through a user interface module.

One skilled in the art will readily appreciate that the invention is well adapted to carry out the objects and obtain the ends and advantages mentioned, as well as those inherent therein. The embodiments described herein are not intended as limitations on the scope of the invention.

### BRIEF DESCRIPTION OF THE DRAWINGS

To facilitate an understanding of the invention, there are illustrated in the accompanying drawings the preferred embodiments from an inspection of which when considered in connection with the following description, the invention, its construction and operation and many of its advantages would be readily understood and appreciated.

FIG. 1a is a diagram illustrating a first embodiment of the system of the present invention that accommodates gaseous fluid.

FIG. 1b is a diagram illustrating a second embodiment of the system of the present invention that accommodates liquid fluid.

FIG. 2 is a diagram illustrating a detailed block diagram of the computer of the computerised duct.

FIG. 3a is a diagram illustrating a first example of the “Main Page” user interface that is displayed on the human-machine interface unit, which pertains to the first embodiment of the present invention.

FIG. 3b is a diagram illustrating a second example of the “Main Page” user interface that is displayed on the human-machine interface unit, which pertains to the second embodiment of the present invention.

FIG. 4 is a diagram illustrating an example of the “Settings” user interface that is displayed on the human-machine interface unit.

FIG. 5 is a diagram illustrating an example of the “Logger” user interface that is displayed on the human-machine interface unit.

FIG. 6.1 is a flowchart illustrating an A-series operational flow pertaining to the first embodiment of the present invention.

FIG. 6.2 is a flowchart that continues from the flowchart of FIG. 6.1.

FIGS. 7.1, 7.2, 7.3, 7.4 and 7.5 are a series of diagrams showing the flow of the fluid within the first embodiment as per the A-series operational flow described in FIGS. 6.1 and 6.2.

FIG. 8.1 is a flowchart illustrating a B-series operational flow pertaining to the second embodiment of the present invention. FIG. 8.2 is a flowchart that continues from the flowchart of FIG. 8.1.

FIGS. 9.1, 9.2, 9.3, 9.4, 9.5, 9.6 and 9.7 are a series of diagrams showing the flow of the fluid within the second embodiment as per the B-series operational flow described in FIGS. 8.1 and 8.2.

FIG. 10 is a flowchart illustrating a series of steps done by a remote computer for providing predictive maintenance of the system of the present invention.

### DETAILED DESCRIPTION OF THE INVENTION

The present invention relates to a system and a method for monitoring the purity of fluid through the creation of an environment that regulates the pressure of the fluid. The invention may also be presented in a number of different embodiments with common elements. According to the concept of the invention, the system comprises a computerised duct connected to a fluid source. Fluid from the fluid source is sampled by having a fluid portion of the fluid source directed into the duct as a sample. The duct is of an environment in which the pressure of the fluidal sample is regulated, so that its purity sensor measures a purity level of the sample, which corresponds to the purity level of fluid flowing within the fluid source.

It should be noted that in the context of the present invention, the term “fluid” preferably refers to materials or substances that exist in a phase that exhibits the behaviour of flowing from one point to another, either through natural means or man-made means. Phases of matter in which the material or substance may exist thereby shall include liquid, gas, plasma, semisolid, or mixtures thereof. Moreover, the term “fluid” may further refer to substances that exhibit behaviours that include fluidity, viscosity, pseudo-plasticity, viscoelasticity, Bingham plasticity, rheopecticity, thixotropy, dilatanticity, or the like. In particular, throughout this description, the term “fluid” shall primarily refer to carbon dioxide being in the gaseous phase or liquid phase.

From here on, it will be understood that, although the terms first, second, etc. are used herein to describe various elements, components, regions, layers or sections, these elements, components, regions, layers or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

The invention will now be described in greater detail, by way of example, with reference to the figures.

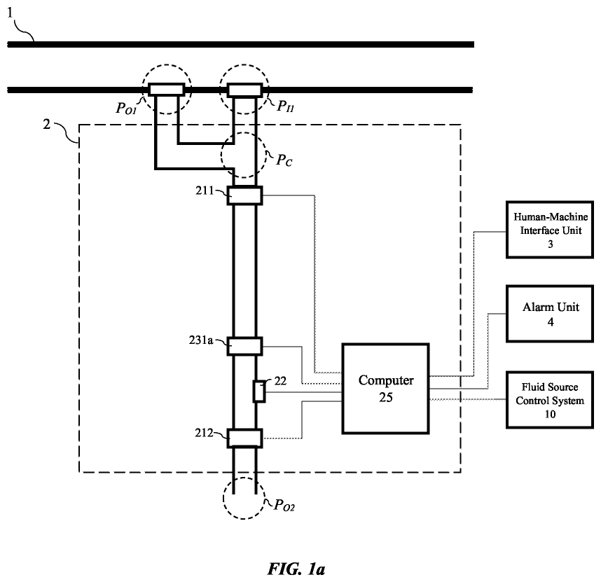

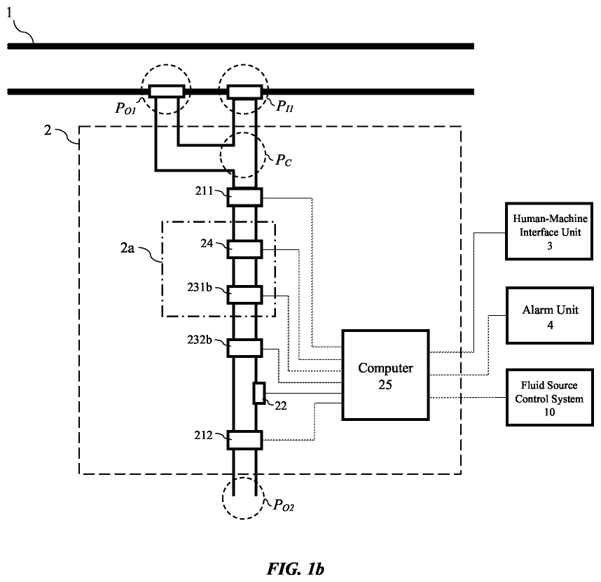

FIG. 1a and FIG. 1B are diagrams illustrating examples of the system of the present invention in different embodiments. In particular, FIG. 1a illustrates the system in its first embodiment A, which accommodates gaseous fluid coming from a fluid source 1. Whereas, FIG. 1b illustrates the system in its second embodiment B which accommodates liquid fluid coming from the fluid source 1. It is noted that both the embodiments A and B possess similarities and differences, which shall now be described below.

Referring to FIG. 1a, the system in its first embodiment A comprises a computerised duct 2 connected to a fluid source 1. Here, the fluid source 1 has the gaseous fluid, preferably being gaseous carbon dioxide, flowing therein. The duct 2 further includes one or more valves, at least one purity sensor 22, a pressure regulator 231a and a computer 25. The one or more valves include a first valve 211 and a second valve 212.

Referring to FIG. 1B, the system in its second embodiment B comprises a computerised duct 2 connected to a fluid source 1. Here, the fluid source 1 has liquid fluid, preferably being liquid carbon dioxide, flowing therein. The duct 2 further includes one or more valves, at least one purity sensor 22, one or more pressure regulators, a phase conversion unit 24 and a computer 25. The one or more valves include a first valve 211 and a second valve 212. The one or more pressure regulators include a first pressure regulator 231b and a second pressure regulator 232b.

Referring to both FIG. 1a and FIG. 1B, both embodiments A and B also involve a human-machine interface (HMI) unit 3, an alert unit 4, and a fluid source control system 10.

Referring to both FIG. 1a and FIG. 1B, in both embodiments A and B, the fluid source 1 is in the form of a pipeline that delivers fluid from an origin to an intended destination through natural or man-made means. Alternatively, the fluid source 1 may be a tank whereby fluid is stored therein, with the tank preferably being pressurized to have an internal pressure that is higher than the atmospheric pressure.

Referring to both FIG. 1 and FIG. 1B, in both embodiments A and B, the duct 2 itself comprises one or more interconnected hollow structures. Composition wise, they are preferably metallic material or any kind of rigid material. Moreover, it is much preferred that each of these structures are elongated and have an inner diameter that is between 6 mm and 7 mm. These structures define the passages of the duct 2 through which the sample travels.

Within both embodiments A and B, the duct 2 further includes a first inlet point P_{I1}, a first outlet point P_{O1}, a convergence point P_{C}, and a second outlet point P_{O2}.

In particular, the first inlet point P_{I1} and the first outlet point P_{O1} are points of the duct 2 that are substantially attached to the body of the fluid source 1. The means by which the first inlet point P_{I1} and the first outlet point P_{O1} are attached may be through welding. Alternatively, the body of the fluid source 1 has built-in sockets that would allow the first inlet point P_{I1} and the first outlet point P_{O1} to be attached thereto.

In particular, the convergence point P_{C} is a point along the duct 2 whereby a delivery structure extending from the first inlet point P_{I1} and a return structure extending from the first outlet point P_{O1} meet and converge. This defines a looped configuration that is present within both embodiments A and B.

In particular, the second outlet point P_{O2} is a point along the duct 2 whereby the sample is purged therefrom.

Referring to both FIG. 1a and FIG. 1B, in both embodiments A and B, the first valve 211 and the second valve 212 are to be in an open state or a shut state. It is to be noted that the section between the first valve 211 and second valve 212 defines a monitoring section of the duct 2.

Preferably, the valves 211 and 212 are to be in an open state or a shut state in a timed manner such that (i) a fluid portion from the fluid source 1 enters the monitoring section of the duct 2 as a sample, (ii) the sample is sufficiently pressure-regulated and for its purity level to be measured by the purity sensor 22, and (iii) the sample is purged from the duct 2 to the external environment.

The valves 211 and 212 may be, but shall not be limited to, mechanical valves, solenoid valves, diaphragm valves, or the like. Preferably, the valves 211 and 212 are at least automated and can be remotely controlled to be in an open state or a shut state.

Referring to both FIG. 1a and FIG. 1B, in both embodiments A and B, the purity sensor 22 is a kind of sensor that measures a purity level based on the concentration of one or more constituents present within the sample. Conversely, it is a kind of sensor that measures the purity level based on the concentration of one or more contaminants present within the sample. Preferably, the purity sensor 22 is capable of identifying and differentiating constituents within the fluid whether they be in a solid, liquid or gas phase. Preferably, the purity sensor 22 operates in an environment that has a pressure equivalent to the atmospheric pressure of 1 bar. The purity sensor 22 may be an optical gas sensor, electrochemical gas sensor, photoionization gas sensor, metal oxide gas sensor or the like.

Referring to both FIG. 1a and FIG. 1B, in both embodiments A and B, the pressure regulators 231a, 231b and 232b regulate the pressure of the sample at points along the monitoring section to be between 0.9 bar to 5 bar, but preferably 1 bar or 14.5 PSI. Since the purity sensor 22 is to measure a purity level of a sample that may have higher pressure than the atmospheric pressure, it is desirable that the pressure regulators 231a, 231b and 232b regulate the pressure within the monitoring section so that the purity sensor 22 may operate in an optimal manner. Moreover, it is preferred that the pressure regulators are capable of regulating up to a pressure of 5 bar so as to cause the sample to be purged out from the monitoring section when required. Even so, the sample may still be purged out at the preferable pressure of 1 bar. The kind of pressure regulator employed in both embodiments A and B may be pressure reducing regulators, back-pressure regulators, or the like. Preferably, the pressure regulators 231a, 231b, and 232b are at least automated and can be controlled to vary the pressure to be regulated within the duct 2.

Referring to FIG. 1B, in the second embodiment B, the phase conversion unit 24 is to convert, or vaporise, a sample from a liquid phase into a gaseous phase. This is so that purity of the fluid may be measured by the purity sensor 22 later on. The kind of phase conversion unit employed in the second embodiment B may be a heater, evaporator device, two-phase heat exchanger, or the like. Preferably, the phase conversion unit 24 is at least automated and can be remotely monitored and regulated.

Referring to both FIG. 1a and FIG. 1B, in both embodiments A, B, the computer 25 is to (i) manage the operations of the valves 211, 212, pressure regulators 231a, 231b, 232b, and the phase conversion unit 24, (ii) interpret the sensor data signals generated by the purity sensor 22, and (iii) generate outputs based on the interpreted sensor data. To this end, it is preferably interfaced with the valves 211, 212, the purity sensor 22, the pressure regulators 231a, 231b, 232b, and the phase conversion unit 24. It is preferably further interfaced with the human-machine interface 3, the alert unit 4 and the fluid source control system 10. Embodiments of the computer 25 may be, but shall not be limited to a motherboard, a single-board computer, a single-board microcontroller, or the like.

The human-machine (HMI) interface 3 is a platform that allows an operator to interact with computerised duct 2 so that the operator may (i) be informed on the current processes that are occurring within the duct 2, (ii) be informed on historical purity levels of the fluid, and (iii) change configurative settings of its computer 25. Preferably, it is in the form of a desktop, laptop, smartphone, or the like. Connections between the computerised duct 2 and the human-machine interface 3 is wired or wireless.

The alert unit 4 is a device or a system that serves to alert an operator when the measured purity level fails to meet a threshold. Means by which the alert unit 4 may alert the operator by producing audio or visual stimuli such as the sounding of an alarm or warning lights. Connections between the duct 2 and the alert unit 4 may be wired or wireless.

The fluid source control system 10 is a system that controls the fluid source 1. In particular, it controls the fluid source in a way that it allows fluid therewithin to flow from one point to another, or prevent the flow of fluid entirely. Connections between the duct 2 and the fluid source control system 10 may be wired or wireless.

Referring to FIG. 1a, the positional arrangement within the first embodiment A is such that (i) the first valve 211 is positioned downstream of the convergence point P_{C}, (ii) the pressure regulator 231 is positioned downstream of the first valve 211, (iii) the purity sensor 22 is positioned downstream of the pressure regulator 231, and (iv) the second valve 212 is positioned downstream of the purity sensor 22 and upstream of the second outlet point P_{O2}.

Referring to FIG. 1B, the positional arrangement within the first embodiment B is such that (i) the first valve 211 is positioned downstream of the convergence point P_{C}, (ii) the phase conversion unit 24 is positioned downstream of the first valve 211, (iii) the first pressure regulator 231b and the second pressure regulator 232b are positioned downstream of the phase conversion unit 24 in succession, (iv) the purity sensor 22 is positioned downstream of the second pressure regulator 232b, and (v) the second valve 212 is positioned downstream of the purity sensor 22 and upstream of the second outlet point P_{O2}.

It is to be noted that the positional arrangements being defined to be “upstream of” or “downstream of” are meant to be interpreted as relative terms.

As for the computer 25, it is at the very least integrated to the duct 2. For example, it is substantially attached or coupled to the body of the hollow structures.

Referring to FIG. 1B, in the second embodiment B, it is seen that the phase conversion unit 24 and the first pressure regulator 231b may be part of a first accessory 2a. More specifically, the second embodiment B may arise from the first embodiment A by having this modular first accessory 2a fitted onto the computerized duct 2 of the first embodiment A in a customizable matter, preferably between the first valve 211 and the pressure regulator 231a.

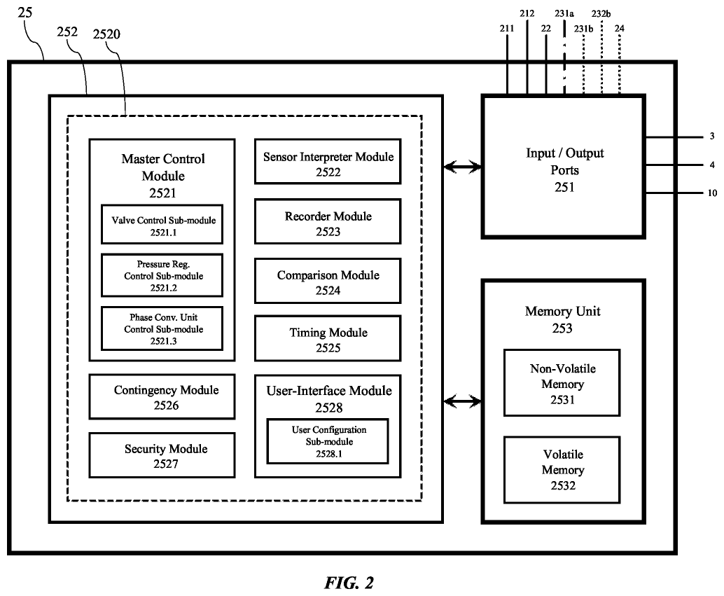

FIG. 2 illustrates a detailed block diagram of the computer 25. In particular, it is noted that the computer 25 has a set of input/output (I/O) ports 251, at least one processor 252 running an application software 2520, and a memory unit 253. In particular, the computer 25 is able to be configured to accommodate the first embodiment A and the second embodiment B.

Regarding the input/output (I/O) ports 251, it is a collective set of terminals that allow the computer 25 to interact with (i) the valves 211 and 212, pressure regulators 231a, 231b and 232b, and phase conversion unit 24 by sending out control signals, (ii) the purity sensor 22 for receiving sensor data signals, (iii) the human-machine interface 3 for a user interface to be displayed thereon, and (iv) the alert unit 4 and the fluid source control system 10 when the measured purity level fails to meet a threshold. These I/O ports 251 may be conventional communication protocols such as Universal asynchronous receiver-transmitter (UART), Serial Peripheral Interface (SPI), Inter-Integrated Circuit (I2C), or the like.

Regarding the processor 252, it performs the scheduling and the execution of software instructions or computer logic instructions based on the application software 2520 pertaining to (i) the control of the valves 211 and 212, pressure regulators 231a, 231b, and 232b, and phase conversion unit 24, and (ii) and measurement of the purity level. Preferably, it is a conventional processor, application-specific integrated circuit (ASIC), a field-programmable gate array (FPGA), or a combination thereof.

Regarding the memory unit 253, it further comprises one or more non-volatile memory units 2531, one or more volatile memory units 2532, or a combination thereof. The non-volatile memory unit 2531 would store program files related to the application software 2520, while volatile memory unit 2532 would temporarily store processing data during runtime of the application software 2520. The non-volatile memory unit 2531 may be, but will not be limited to, data storage devices such as hard disk drives (HDD), solid-state drives (SSD), hybrid drives, secure digital (SD) cards, TransFlash (TF) cards or their likes. The volatile memory unit 2532 may be, but will not be limited to, dynamic random access memory (DRAM), static dynamic random access memory (SRAM), and their synchronous variants.

It is further shown in FIG. 2 that processor 252 operates one or more modules. More particularly, these modules are software modules of the application software 2520 that operates, or runs, on the processor 252 for computing algorithms and generating control instructions. These modules include a master control sub-module 2521, a sensor interpreter module 2522, a recorder module 2523, a comparison module 2524, a timing module 2525, a contingency module 2526, a security module 2527, and a user interface module 2528. It should be noted that the presented modules need not be in a software embodiment, and may be a hardware embodiment where they are connected to the processor 252 or they are their own independent computer system. Ancillary modules may be included to provide support for the aforementioned modules.

In particular, the master control module 2521 serves to generate control signals for controlling the operations of any one or a combination of the valves 211, 212, the pressure regulators 231a, 231b, 232b, or the phase conversion unit 24. Preferably, it further comprises one or more sub-modules which include a valve control sub-module 2521.1, a pressure regulator sub-module 2521.2, and a phase conversion unit control sub-module 2521.3.

The valve control sub-module 2521.1 generates control signals for controlling the valves 211 and 212 to be either in an open state or shut state. The pressure regulator sub-module 2521.2 shall generate control signals for controlling the pressure regulators 231a, 231b, 232b for them to exert a specified pressure. The phase conversion unit control sub-module 2521.3 generates control signals for controlling the phase conversion unit 24 for them to provide a specified temperature that (i) brings the fluid to its vaporisation point so that liquid fluid may be converted into a gaseous phase, and (ii) maintains the fluid to be above its dew point.

In particular, the sensor interpreter module 2522 serves to convert the sensor data signals received from the sensor module, through the I/O Ports 251 into an interpreted sensor data that represents the measured purity level in a numerically comprehensible manner. To do so, it runs one or more proprietary codes for generating the interpreted sensor data.

In particular, the recorder module 2523 serves to record the measured purity level and store it in the memory unit 253. Besides the interpreted sensor data, the recorder module 2523 may also record (i) a timestamp indicating when the sensor measurement was taken, (ii) the current status of the system, and (iii) users who have logged into the system.

In particular, the comparison module 2524 serves to compare the measured purity level with a threshold. For the case of the invention, there may be a default threshold as defined by the program files, or it may be re-configured by the operator. Preferably, the threshold is adjusted based on the intended use case of the fluid as the required purity of the fluid may vary for different use cases. Depending on the outcome of the comparison, the comparison module 2524 shall inform the contingency module 2526 on the results of the comparison.

In particular, the timing module 2525 serves to dictate operational frequencies of the valves 211 and 212, in conjunction with the master control module 2521. It shall dictate (i) the frequency in which the first valve 211 switches between an open state and a shut state, and/or (ii) the frequency in which the second valve 212 switches between an open state and a shut state.

Moreover, the timing module 2525 manages these operation periods such that (i) the sample entering the monitoring section is of a small volume relative to said section, (ii) the purity sensor 22 has ample time to measure the purity level of the sample, and (iii) equilibrium vapour pressure is prevented from being achieved within the space of the monitoring section. Should it be required that the second valve 212a assume a shut state, it is preferred that ample time is provided for the space within the monitoring section to return to being a partial vacuum after the sample is purged into the environment.

In particular, the contingency module 2526 serves to generate control signals relevant to the alert unit 4 and the fluid source control system 10 when it is informed by the comparison module 2524 on the results of the comparison. These control signals are instructions for (i) the alert unit 4 to begin alerting the operator through both one or both audio or visual stimuli, and (ii) the fluid source control system 10 to shut off the fluid source 1 to prevent fluid flowing therein from reaching its intended destination.

In particular, the security module 2527 serves to provide a layer of security to the system. It shall enforce authentication measures by requesting a username and password when the operator attempts to make changes to system configuration. Encrypted usernames and passwords may be stored within the memory unit 253, and the security module 2527 shall run cryptographic hash functions to verify that the username and password keyed into the system match.

In particular, the user interface module 2528 serves to compile either one or both real-time data and historical data that has been stored in the memory unit 253 into a format that may be displayed on the HMI unit 3 as a Graphical User Interface (GUI). Also, the user interface module 2528 shall compile the data in such a way that it is displayed on the HMI unit 3 as three or more distinct interfaces. These interfaces include a Main Page interface, a Settings interface and a Logger interface. Furthermore, the user interface module 2528 further includes a user configuration sub-module 2528.1.

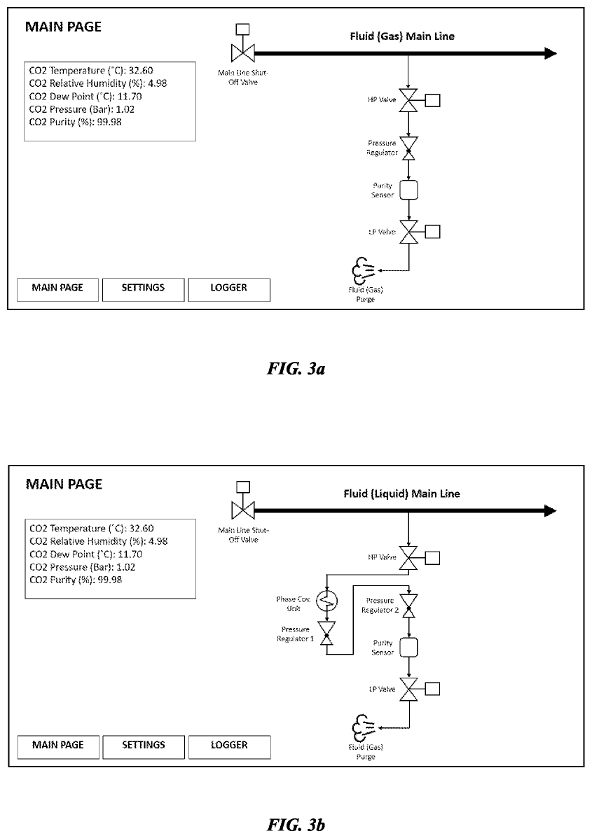

A first Main Page interface corresponding to the first embodiment A is as shown in FIG. 3a. A second Main Page interface corresponding to the second embodiment B is as shown in FIG. 3b. Both Main Page interfaces are to display an interactive schematic diagram of the system. Included are (i) symbolic indicators of the valves 211 and 212, the purity sensor 22, the pressure regulators 231a, 231b, and 232b, and the phase conversion unit 24, (ii) the positional relation between the aforementioned items, (iii) indicative depictions of the sample travelling down the duct 2, and (iv) current sensor data of the system, which includes the measured purity level of the sample. In a first usage example, when the sample is purged from the duct 2, the “Fluid (Gas) Purge” indicator will light up. In a second usage example, the operator directly selects any one of the symbolic depictions to override system control.

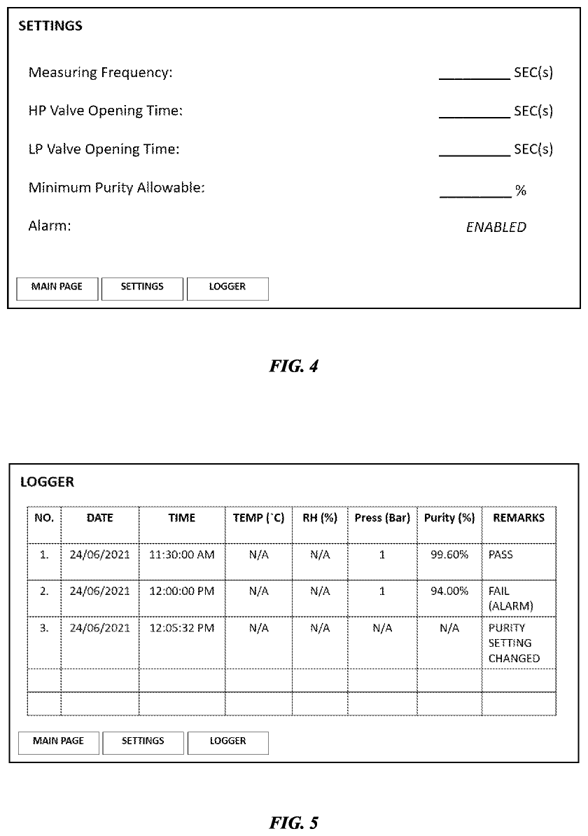

A Settings interface is as shown in FIG. 4. It displays one or more settings in which the operator can interact therewith for configuring the system. In particular, the user configuration sub-module 256.1 facilitates the input of information from the operator through an input device of the HMI unit 3. The parameters available for the operator to configure preferably include (i) data logging frequency, (ii) period in which the first valve 211 switches between an open state and a shut state, (iii) period in which the second valve 212 switches between an open state and a shut state, (iv) fluid purity threshold in a 2 decimal point percentage, (v) enablement of interface with the alert unit 4. It is inferable that further advanced embodiments would have more parameters for the operator to configure.

A Logger interface is as shown in FIG. 5. It displays a table that lists out data pertaining to the status of the system at various periods of time. In particular, the Logger interface updates the table (i) at specified time intervals, (ii) in the event where the measured purity level fails to meet the threshold, or (iii) in the event where configurations have been made to the system. The table shall present the data in a reverse chronological order.

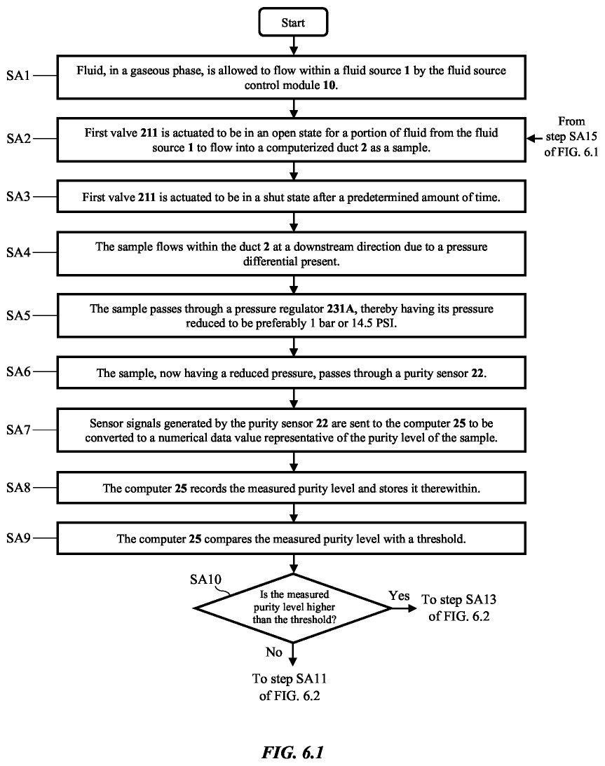

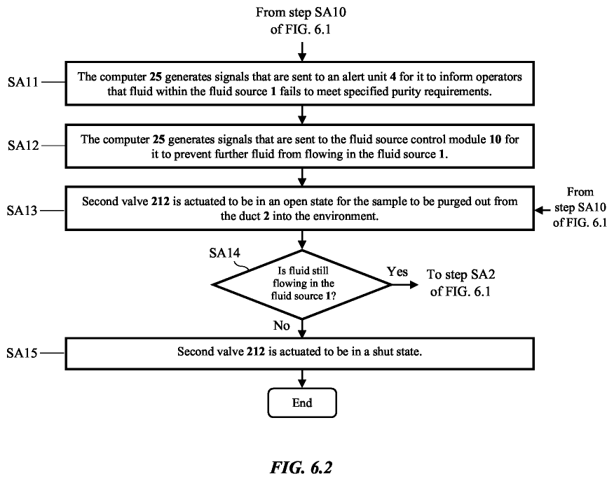

FIGS. 6.1 and 6.2 illustrate an A-series operational flow pertaining to the system of the present invention in its first embodiment A. The system, in its first embodiment A, is to perform a series of steps in which gaseous fluid from the fluid source 1 is directed into the computerised duct 2 to have its purity level measured by the purity sensor 22.

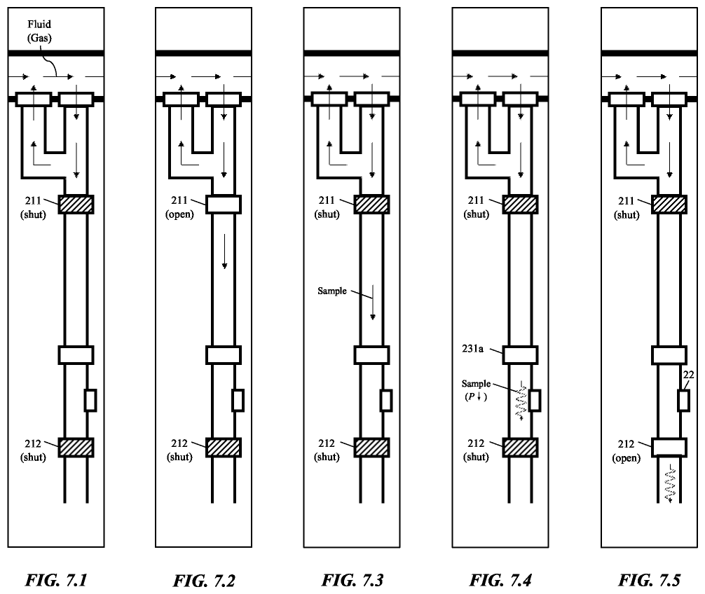

FIGS. 7.1 to 7.5 are a series of figures depicting the flow of fluids within this first embodiment A as per the A-series operational flow, and it is to be understood that the valves 211, 212, sensor unit 22, and pressure regulators 231a are interfaced with the computer 25.

Preferably, it is assumed that the initial conditions of the computerised duct 2 are such that (i) the valves 211 and 212 are in a shut state, and (ii) the monitoring section, defined to be the section between the valves 211 and 212, is at least a partial vacuum, and (iii) the computer 25 has been set up with the sensor 22 being in a ready mode.

The steps illustrated in FIGS. 6.1 and 6.2 shall now be described with reference to FIGS. 7.1 to 7.5.

First, in step SA1, fluid, in a gaseous phase, is allowed to flow within a fluid source 1 to travel from an origin to an intended destination by the fluid source control module 10.

During this step SA1, fluid from the fluid source 1 may enter the duct 2 through the first inlet point P_{I1} and exit through the first outlet point P_{O1} as (i) the first valve 211 in a shut state, and (ii) due to the looped configuration. The fluid returns to the fluid source 1. This is illustrated in FIG. 7.1.

Next, in step SA2, under the control of the valve control module 2521.1 and timing module 2525, the first valve 211 is actuated to be in an open state for a fluid portion from the fluid source 1 to flow into a monitoring section of the computerised duct 2 as a sample. This is illustrated in FIG. 7.2.

Next, in step SA3, under the control of the valve control module, the first valve 211 is actuated to be in a shut state after a predetermined amount of time.

During this step SA3, it should be noted that fluid shall still continuously enter the first inlet point P_{I1}. As the first valve 211, being positioned after the convergence point P_{C}, is in a shut state, there will be a pressure build-up occurring at the first valve 211. However, due to the looped configuration, a fresh flow of fluid shall continuously be situated adjacent to the first valve 211. Moreover, pressure build-up at the first valve is relieved as the fluid is redirected to return to the fluid source 1 through the first outlet point P_{O1}. As such, the first valve 211 is relieved from experiencing significant pressure build-up when it is actuated from being in an open state to a shut state, thereby improving its operational lifespan and integrity.

Next, in step SA4, the sample flows within the duct 2 in a downstream direction. Preferably, the entry of the sample into this section creates a pressure differential that causes the sample to flow like so. An illustration of the flow of fluids in steps SA3 and SA4 is shown in FIG. 7.3.

Next, in step SA5, the sample passes through a pressure regulator 213a, thereby having its pressure reduced to be between 0.9 bar to 5 bar, but preferably 1 bar or 14.5 PSI. The sample, now having a reduced pressure, continues to flow in the downstream direction due to the pressure differential still present.

Next, in step SA6, the sample, now having reduced to a desired pressure of preferably 1 bar or 14.5 PSI, passes through purity sensor 22, or at least the detectors of the purity sensor 22. With this, the purity sensor 22 measures the purity of the sample and generates sensor data signals therefrom. This is illustrated in FIG. 7.4.

Next, in step SA7, the sensor data signals are then sent to the computer 25 and its sensor interpreter module 25 shall convert it to a numerical value representative of the purity level of the sample.

Next, in step SA8, the computer 25, more particularly its recorder module 2523, shall record the purity level and stores it within the memory unit 253 of the computer 25.

Next, in step SA9, the computer 25, more particularly its comparison module 2524, shall obtain the purity level either from the recorder module 2523 or the memory unit 253 of the computer. It shall then compare the measured purity level with a threshold, this threshold being a default threshold or a configured threshold.

The next step, step SA10 is a decision step. Here, it shall be determined whether or not the measured purity level is higher than the threshold. Should this not be the case, step SA10 proceeds to step SA11. Should this be the case, step SA10 proceeds to step SA13.

In step SA11, the computer 25, more particularly the contingency module 2526 is informed that the measured purity level of the sample fails to be higher than the threshold. The contingency module 2526 shall interpret that the purity level of the fluid in the fluid source 1 fails to meet specified purity requirements for its use case. As such, it shall generate signals that are to be sent to the alert unit 4 to inform operators that the fluid within the fluid source 1 fails to meet specified purity requirements.

Following step SA11 is step SA12, whereby the computer 25, more particularly the contingency module 2526, shall also generate signals that are sent to the fluid source control module 10 to inform it to prevent further fluid from flowing within the fluid source 1 so that the impure fluid does not reach its intended destination.

Step SA13 is a shared step that follows from either one of step SA10 or step SA12. Here, under the control of the valve control module 2521.1 and timing module 2525, the second valve 212 is actuated to be in an open state for the sample to be purged out from the duct 2 into the environment. This is illustrated in FIG. 7.5.

Following step SA13 is step SA14, which is a decision step. Here, it shall be determined by the computer 25 whether or not the fluid source control module 10 still allows fluid to flow within the fluid source 1.

Should this be the case, it means that fluid is still flowing within the fluid source 1. As such, step SA14 shall return to step SA2 in which operations pertaining to the measurement of the purity level of the fluid continues. It is to be noted that in succeeding step cycles, the second valve 212 remains in an open state so that the sample is instantaneously purged from the monitoring section after its purity level is measured. More specifically, in succeeding step cycles, FIGS. 7.1 to 7.5 still apply, but with the second valve 212 remaining in an open state for an entered sample to continuously flow out into the environment after having its purity level measured.

Should this not be the case, it means that the fluid source control module 10 has prevented fluid from flowing within the fluid source 1, which may be caused by (i) the computer 25 previously informing it that the sample had a purity level that failed to meet the threshold, or (ii) direct intervention by the operator through the HMI unit 3.

With this, step SA14 proceeds to step SA15, whereby under the control of the valve control module 2521.1 and timing module 2525, the second valve 212 is actuated to be in a shut state. Preferably, at this point, the monitoring section returns to being at least a partial vacuum once more, and operations pertaining to the measurement of the purity level of the fluid come to an end.

With this, the A-series operational flow pertaining to the system of the present invention in its first embodiment A has been sufficiently elucidated.

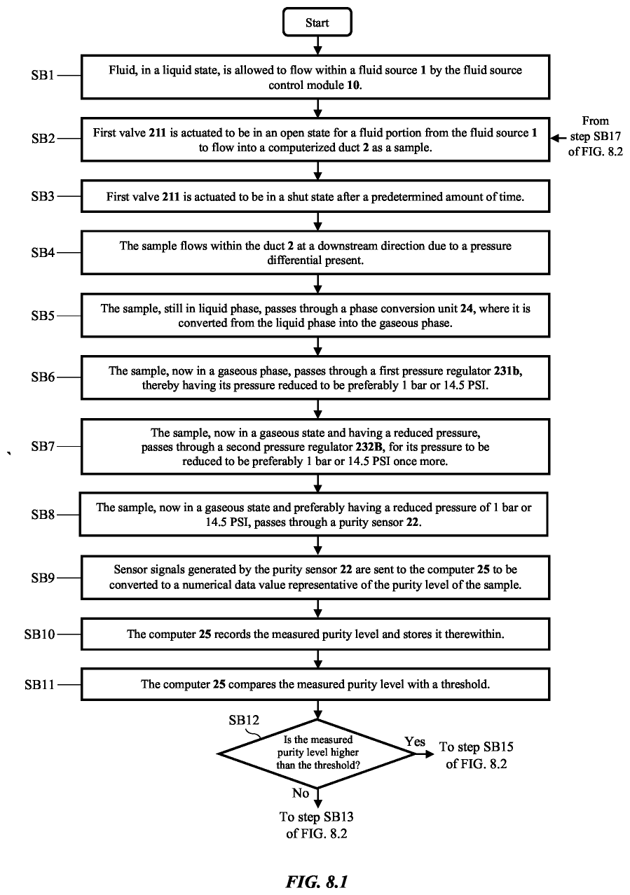

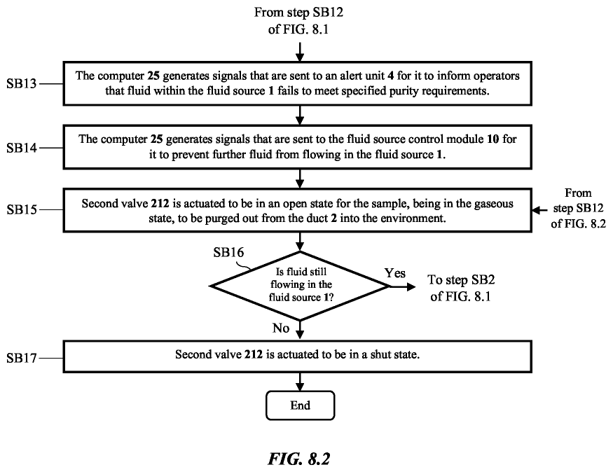

FIGS. 8.1 and 8.2 illustrate a B-series operational flow pertaining to the system of the present invention in its second embodiment B. The system, in its first embodiment B, is to perform a series of steps in which liquid fluid from the fluid source 1 is directed into the computerised duct to have its purity level measured by the purity sensor 22.

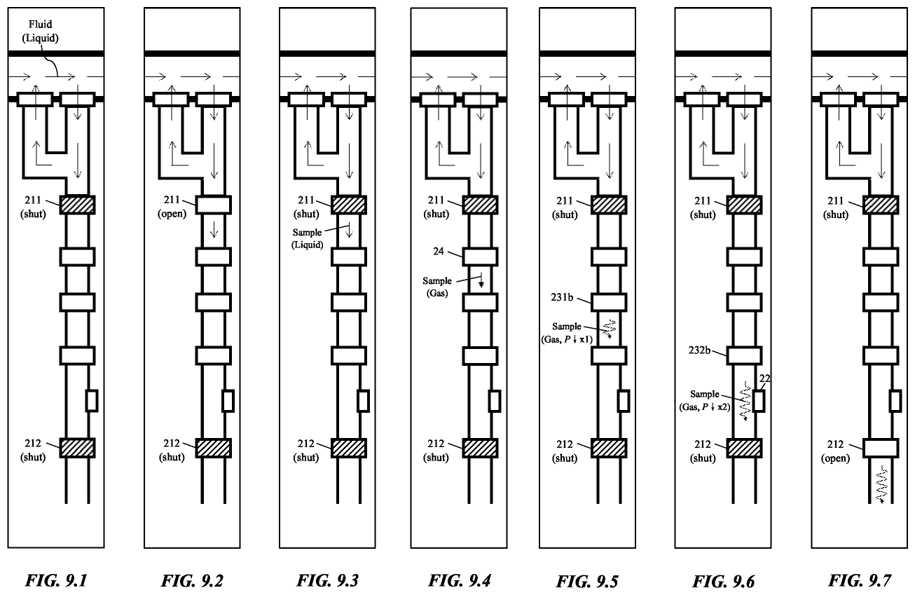

FIGS. 9.1 to 9.7 are a series of figures depicting the flow of fluids within this first embodiment B as per the B-series operational flow, and it is to be understood that the valves 211 and 212, the sensor unit 22, the pressure regulators 231b and 232b, and the phase conversion unit 24, are interfaced with the computer 25.

Preferably, it is assumed that the initial conditions of the computerised duct 2 are such that (i) the valves 211, 212 are in a shut state, (ii) the monitoring section, defined to be the section between the valves 211 and 212, is at least a partial vacuum, and (iii) the phase conversion unit 24 is ready for phase conversion of the fluid.

The steps illustrated in FIGS. 8.1 and 8.2 shall now be described with reference to FIGS. 9.1 to 9.7.

First, in step SB1, fluid, in a gaseous phase, is allowed to flow within a fluid source 1 to travel from an origin to an intended destination by the fluid source control module 10.

During this step SB1, similar to step SA1 as before, fluid from the fluid source 1 may enter the duct 2 through the first inlet point P_{I1} and exit through the first outlet point P_{O1} due to (i) the first valve 211 assuming a shut state, and (ii) due to the looped configuration. The fluid returns to the fluid source 1. This is illustrated in FIG. 9.1.

Next, in step SB2, under the control of the valve control module 2521.1 and timing module 2525, the first valve 211 is actuated to be in an open state for a fluid portion from the fluid source 1 to flow into a monitoring section of the computerised duct 2 as a sample. This is illustrated in FIG. 9.2.

Next, in step SB3, under the control of the valve control module, the first valve 211 is actuated to be in a shut state after a predetermined amount of time.

During this step SB3, similar to step SA3 before, fluid shall still continuously enter the first inlet point P_{I1}. As the first valve 211, being positioned after the convergence point P_{C}, is in a shut state, there will be a pressure build-up occurring at the first valve 211. However, due to the looped configuration, this pressure build-up is relieved as the fluid is redirected to return to the fluid source 1 through the first outlet point P_{O1}. As such, the first valve 211 is relieved from experiencing significant pressure build-up when it is actuated from being in an open state to a shut state, thereby improving its operational lifespan and integrity.

Next, in step SB4, the sample flows within the duct 2 in a downstream direction. Preferably, the entry of the sample into this section creates a pressure differential that causes the sample to flow like so. An illustration of the flow of fluids in steps SB3 and SB4 are shown in FIG. 9.3.

Next, in step SB5, the sample passes through the phase conversion unit 24. Here, the phase conversion unit 24 shall convert the sample from being in a liquid phase into a gaseous phase. The sample, now in a gaseous phase, has an increase in kinetic energy and hence continues flowing in a downstream direction due to an increase in the pressure differential. This is illustrated in FIG. 9.4.

Next, in step SB6, the sample, now in a gaseous phase, passes through a first pressure regulator 231b, thereby having its pressure reduced to be between 0.9 bar to 5 bar, but preferably 1 bar or 14.5 PSI. This is illustrated in FIG. 9.5. The sample, now having a reduced pressure, continues to flow in the downstream direction due to the pressure differential.

Next, in step SB7, the sample, now in a gaseous phase and having reduced pressure, passes through a second pressure regulator 231b, thereby having its pressure reduced once again to be between 0.9 bar to 5 bar, but preferably 1 bar or 14.5 PSI. The reason for going through the second pressure regulator 232b which provides the same pressure regulation is to prevent either one of the pressure regulators from freezing out due to the temperature of the liquid fluid. Moreover, such a configuration shall ensure that the entire gaseous sample has a pressure of preferably 1 bar. The sample, now having a reduced pressure, continues to flow in the downstream direction due to the pressure differential.

Next, in step SB8, the sample, now in a gaseous phase and preferably having a desired pressure of 1 bar or 14.5 PSI, passes through purity sensor 22, or at least the detectors of the purity sensor 22. With this, the purity sensor 22 measures the purity of the sample and generates sensor data signals therefrom. This is illustrated in FIG. 9.6.

Next, in step SB9, the sensor data signals are then sent to the computer 25 and its sensor interpreter module 25 shall convert it to a numerical value representative of the purity level of the sample.

Next, in step SB10, the computer 25, more particularly its recorder module 2523, shall record the purity level and stores it within the memory unit 253 of the computer 25.

Next, in step SB11, the computer 25, more particularly its comparison module 2524, shall obtain the measured purity level either from the recorder module 2523 or the memory unit 253 of the computer. It shall then compare the measured purity level with a threshold, this threshold being a default threshold or a configured threshold.

The next step, step SB12 is a decision step. Here, it shall be determined whether or not the measured purity level is higher than the threshold. Should this not be the case, step SB12 proceeds to step SB13. Should this be the case, step SB12 proceeds to step SB15.

In step SB13, the computer 25, more particularly the contingency module 2526 is informed that the measured purity level of the sample fails to be higher than the threshold. The contingency module 2526 shall interpret that the purity level of the fluid in the fluid source 1 fails to meet specified purity requirements for its use case. As such, it shall generate signals that are to be sent to the alert unit 4 to inform operators that the fluid within the fluid source 1 fails to meet specified purity requirements.

Following step SB13 is step SB14, whereby the computer 25, more particularly the contingency module 2526, shall also generate signals that are sent to the fluid source control module 10 to inform it to prevent further fluid from flowing within the fluid source 1 so that the impure fluid does not reach its intended destination.

Step SB15 is a shared step that follows from either one of step SB12 or step SB14. Here, under the control of the valve control module 2521.1 and timing module 2525, the second valve 212 is actuated to be in an open state for the sample to be purged out from the duct 2 into the environment. This is illustrated in FIG. 9.7.

Following step SB15 is step SB16, which is a decision step. Here, it shall be determined by the computer 25 whether or not the fluid source control module 10 still allows fluid to flow within the fluid source 1.

Should this be the case, it means that fluid is still flowing within the fluid source 1. As such, step SB16 shall return to step SB2 in which operations pertaining to the measurement of the purity level of the fluid continues. It is to be noted that in succeeding step cycles, the second valve 212 remains in an open state so that the sample is instantaneously purged from the monitoring section after its purity level is measured. More specifically, in succeeding step cycles, FIGS. 9.1 to 9.7 still apply, but with the second valve 212 remaining in an open state for an entered sample to continuously flow out into the environment after having its purity level measured.

Should this not be the case, it means that the fluid source control module 10 has prevented fluid from flowing within the fluid source 1, which may be caused by (i) the computer 25 previously informing it that the sample had a purity level that failed to meet the threshold, or (ii) direct intervention by the operator through the HMI unit 3.

With this, step SB16 proceeds to step SB17, whereby under the control of the valve control module 2521.1 and timing module 2525, the second valve 212 is actuated to be in a shut state. Preferably, at this point, the monitoring section returns to being at least a partial vacuum once more, and operations pertaining to the measurement of the purity level of the fluid come to an end.

With this, the B-series operational flow pertaining to the system of the present invention in its second embodiment B has been sufficiently elucidated.

While it is illustrated and elucidated that, in the A-series operational flow and the B-series operational flow, the second valve 212 remains in an open state in succeeding step cycles and only assumes a shut state during the end of the operation, it may not necessarily be the case. Alternatively, the second valve 212 may periodically alternate between an open state and a shut state in each step cycle. More specifically, the second valve may assume an open state for the sample to be purged into the environment, and may assume a shut state after the sample is purged into the environment.

In a first example of an advanced embodiment of the present invention, pump units may be included in the vicinity of the first inlet point P_{I1}, the first outlet point P_{O1}, and the second outlet point P_{O2}. These pump units are devices that direct the flow of fluid through man-made means, such as impellers, fans, of the like. To further clarify this, there may be a first pump unit at the first inlet point P_{I1} that draws fluid from the fluid source 1 into the duct 2, a second pump unit at the first outlet point P_{O1} that draws the fluid from the duct 2 back into the fluid source 1, and a third pump unit at the second outlet point P_{O2} that draws the sample from the duct 2 for purging it into the environment.

In a second example of an advanced embodiment of the present invention, extra sensors may be present for further monitoring. To further clarify this, the system of the present invention may further include sensors such as a temperature sensor, a pressure sensor and a relative humidity sensor. The temperature sensor measures the temperature within the monitoring section for ensuring the vaporisation point and dew point of the fluid are reached. The pressure sensor for monitoring the pressure within the monitoring section. The relative humidity sensor for measuring the relative humidity of the sample. The sensor data measured by these sensors may similarly be interpreted by the sensor interpreted module 2522 and their measured values may be shown on the user interface as shown in the Logger interface of FIGS. 3a, 3b and 5.

In a third example of an advanced embodiment, it is understood that based on the description of the first embodiment A and the second embodiment B, a third embodiment that possesses the features and characteristics may be construed. Moreover, this third embodiment may have additions that include the means to determine whether the fluid flowing in the fluid source 1 is of a gaseous phase or liquid phase, and additional valves to direct fluid to immediately enter the first valve 221 or direct fluid to at least pass through the phase conversion unit 24 before entering the first valve 221. The computer 25 may be programmed accordingly to facilitate the control of these additions.

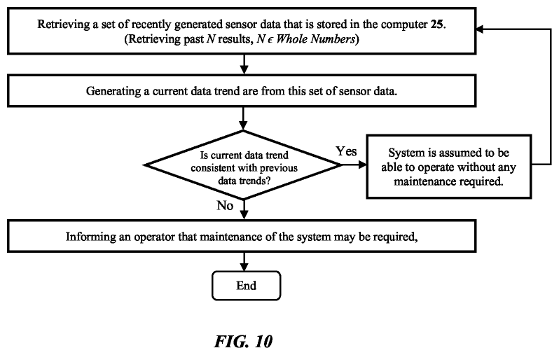

In a fourth example of an advanced embodiment, the computer 25 may be further interfaced with one or more remote computers that serve to provide predictive maintenance of the system. A flowchart illustrating a series of steps done by a remote computer for providing predictive maintenance of the system is shown in FIG. 10. Here, in a first step, a set of recently generated sensor data that is stored in the computer 25 is retrieved. Then, in a second step, a data trend is generated from this set of sensor data. The third step is a decision step whereby it shall be determined whether or not the current data trend is consistent with previous data trends. Should this not be the case, the third step proceeds to a fourth step whereby the operator is informed that maintenance of the system may be required, such as changing of filters of the fluid source 1. Should this be the case, the third step proceeds to a fifth step whereby the system is assumed to be able to operate without any maintenance required.

With this, the details pertaining to the system and the method of the present invention have been sufficiently elucidated.

The present disclosure includes as contained in the appended claims, as well as that of the foregoing description. Although this invention has been described in its preferred form with a degree of particularity, it is understood that the present disclosure of the preferred form has been made only by way of example and that numerous changes in the details of construction and the combination and arrangements of parts may be resorted to without departing from the scope of the invention.

What is claimed is:

1. A system for monitoring purity of fluid, comprising:
a duct connecting to a fluid source, which includes:
a first valve for directing the fluid flowing from the fluid source into the duct, or portions thereof, as a sample;
a purity sensor disposed downstream of the first valve; and
one or more pressure regulators for controlling pressure within the duct, or portions thereof;
wherein the duct, or portions thereof, is of an environment with a desired pressure for measuring a purity level of the sample by the purity sensor.

2. The system according to claim 1, wherein the duct further comprises a phase conversion unit located upstream of the purity sensor for converting the sample from a liquid phase into a gaseous phase.

3. The system according to claim 2, wherein the pressure regulators include any one or a combination of:
a first pressure regulator disposed downstream of the phase conversion unit, for regulating the pressure of the sample after it passes through the phase conversion unit; and
a second pressure regulator disposed upstream of the purity sensor, for regulating the pressure of the sample before it passes through the purity sensor.

4. The system according to claim 1, wherein the desired pressure ranges from 0.9 bar to 5 bar.

5. The system according to claim 1, wherein the duct further comprises a second valve located downstream of the purity sensor for purging the sample therefrom.

6. The system according to claim 5, further comprising at least one processor that operates a combination of modules that include:
a master control module for at least controlling either one or a combination of the first valve, the second valve;
a recorder module for recording the purity level of the sample and storing it in a memory unit; and
a comparison module for comparing the purity level of the sample with a threshold.

7. The system according to claim 6, wherein the processor further operates one or a combination of modules that include:
a timing module, for controlling the frequency of monitoring;
a security module, for protecting the system from unauthorized use; and
a user interface module, for compiling purity levels that were recorded through the recorder module for them to be displayed on a human-machine interface unit.

8. The system according to claim 6, further comprising an alert unit that produces either one or both audio and visual stimuli when the purity level of the sample is below the threshold.

9. The system according to claim 1, wherein the duct further comprises a return structure provided upstream of the first valve and connected upstream of the fluid source that allows fluid that entered the duct to return to the fluid source.

10. The system according to claim 1, wherein the fluid is either one of liquid carbon dioxide or gaseous carbon dioxide.

11. A method for monitoring purity of fluid, comprising the steps of:
directing the fluid from a fluid source into a duct of a system for monitoring the purity of fluid, as a sample; and
measuring a purity level of the sample within the duct by a purity sensor;
wherein the duct, or portions thereof, is of an environment with a desired pressure for measuring the purity level of the sample by the purity sensor.

12. The method according to claim 11, further comprising the step of converting the sample within the duct from a liquid phase into a gaseous phase by a phase conversion unit prior to measuring the purity level of the fluid by the purity sensor.

13. The method according to claim 12, further comprising either one or both the steps of:
regulating the pressure of the sample after it passes through the phase conversion unit, by a first pressure regulator; and
regulating the pressure of the sample before it passes through the purity sensor by a second pressure regulator.

14. The method according to claim 11, wherein the desired pressure ranges from 0.9 bar to 5 bar.

15. The method according to claim 11, further comprising the step of purging the sample from the duct through a second valve upon measuring the purity level of the fluid.

16. The method according to claim 15, further comprising the steps of:
controlling either one or both the first valve and the second valve to open or shut, through a master control module;
recording the purity level of the sample, through a recorder module; and
comparing the purity level of the sample with a threshold, through a comparison module.

17. The method according to claim 16, wherein the step of comparing the purity level of the sample with a threshold further comprises the steps of:
determining that the purity level of the sample is below the threshold;
producing either one or both audio and visual stimuli using an alert unit; and
preventing further flow of the fluid into the fluid source.

18. The method according to claim 16, further comprising the step of compiling the purity levels that were recorded by the recorder module to be displayed on a human-machine interface unit, through a user interface module.
